(19) United States
(12) Patent Application Publication
(10) Pub. No.: US 2006/0135856 A1
(43) Pub. Date: Jun. 22, 2006

(54) Method and apparatus for communicating with a medical device

(76) Inventors: Stefan Breuer, Horb (DE); Rolf Neumann, Calw (DE)

Correspondence Address: PHILIPS INTELLECTUAL PROPERTY & STANDARDS, 595 MINER ROAD, CLEVELAND, OH, 44143, US

(21) Appl. No.: 10/541,560

(22) PCT Filed: Dec. 15, 2003
(86) PCT No.: PCT/IB03/06086

§ 371 Date: Jul. 6, 2005

(30) Foreign Application Priority Data
Jan. 7, 2003 (EP) 03100009.4

(51) Int. Cl. A61B 5/00

(52) U.S. Cl. 600/300; 128/903

(57) ABSTRACT

The invention relates to a method of communicating with a medical device (10), in which an interface (12) is provided to which either measurement means (14) or an external device (16) can be connected and via which measured signals or data are transmitted from the measurement means (14) or the external device (16) to the medical device (10). By virtue of the invention, only one interface is required to use the medical device both in the measurement mode and in the communication mode, in which, for example, a software update can be performed in the medical device via the interface.

The invention relates to a method of communicating with a medical device as claimed in the precharacterizing clause of claim 1 and to a corresponding apparatus as claimed in the precharacterizing clause of claim 8.

Medical devices, such as measurement devices for recording bodily functions, frequently comprise electronics for measuring and recording measured data. The measured data may come from one or more measurement means, in particular sensors. By way of example, mention may be made here of a portable cardiograph for electrocardiography, as is known for example from GB 2 368 127 A.

Such devices may also comprise a microprocessor and software, in particular in order to be able to evaluate the recorded data, for example in order to trigger alarms immediately in the event of life-threatening states. In most devices, the software is stored in a non-volatile, rewritable memory, for example an EEPROM. Many devices of this type also have a communication interface for transmitting recorded data to a host system, for example a personal computer.

FIG. 3 shows a block diagram of a conventional medical device 50 which comprises a host system 52 and a medical measurement device 54. The medical measurement device 54 is connected for communication purposes to the host system 52 via a communication interface 56 and an electrical line 58. Via a sensor interface 60 and an electrical line 62 connected thereto, the medical measurement device 54 can also receive electrical measured signals from a sensor 64 which may be fitted to the body of a patient.

During operation, the measurement device 54 receives, for example, analog electrical measured signals from the sensor 64, converts the received measured signals into digital data and prepares the converted data for further processing by the host system 52. It then transmits the prepared data, via the communication interface 56 and the line 58, to the host system 52, which may comprise a computer, having a memory and a display for recording and displaying the received data, and also a printer.

From time to time, it may be necessary to update an item of software that is stored in the medical measurement device 54 in a non-volatile and writable memory, for example in a Flash EEPROM. This is particularly the case when the software has been reworked in order to eliminate faults or to enable new functions of the measurement device 54. To date, such a software update has been carried out via the communication interface 56.

However, the host system 52 must be configured for the software update, since the software update, that is to say transferring into the measurement device and updating of the software present therein, is essentially controlled from the host system 52. This requires additional technical complexity in the host system 52. In particular, a special protocol is required for the software update, which protocol must be implemented in the host system 52. However, the implementation complexity necessary for this is not in proportion to the use, since a software update need only be carried out on a very irregular and often very infrequent basis.

This implementation is particularly expensive if the medical measurement device is used not just with one but rather with a large number of different host systems, for example if the measurement device is an OEM board which is installed in many different medical devices. In such a case, all the different host systems must implement the complete functionality for the software update. The alternative, namely to construct the measurement device for a software update from the medical device, is not practical for reasons of cost, if it is possible at all.

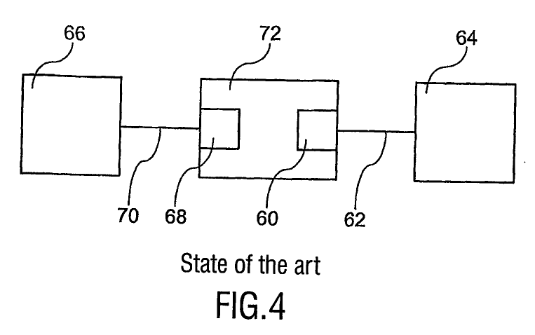

Another possibility for the software update is to use a PC as software update device. As shown in FIG. 4, a PC 66 can be connected for this purpose, by means of an electrical line 70, to an external interface 68 of a medical device 72. The external interface 68 is assigned in the medical device 72 to a host system which can receive data from the PC 66 via said external interface. By way of example, the external interface may be a serial interface in accordance with the RS232C standard or a USB interface. However, considerable technical complexity is also required in this case for the host system in the form of the external interface 68. In addition, the host system must likewise transmit, by means of a special protocol, the software update to a medical measurement device in the medical device 72. Moreover, precautions must be taken to ensure that, during a software update, a patient who is electrically connected to the medical device 72 by way of the sensor 64 is protected against any malfunctions of the device 72 on account of the software update. For this purpose, either it is necessary for the PC 66 or a software update device to be certified and tested in order to be able to use it to carry out a software update while the medical device 72 is in operation, or it must be ensured that the PC 66 or a software update device and the sensor 64 are not connected to the medical device 72 at the same time.

It is therefore an object of the invention to provide a method and an apparatus for communicating with a medical device, which on the one hand require a lower degree of technical complexity than the known methods outlined above and on the other hand ensure that patients are not put at risk in the course of communication, in particular in connection with a software update of the medical device.

This object is achieved by a method having the features as claimed in claim 1 and by an apparatus having the features as claimed in claim 8. Preferred refinements of the invention emerge from the dependent claims.

An essential concept of the invention is to use an interface of the medical device, which interface is provided for one or more sensors, electrodes or similar measurement means for measuring patient signals, also to communicate between medical device and an external device. Communication is in this case understood to mean the communication with another external device, in particular a PC. The advantage of this concept is that the medical device requires essentially just one interface for a measurement mode and communication mode. The technical complexity is therefore only prescribed by one interface which is provided for two different uses. A saving is made with regard to at least one special communication interface as used in the prior art. In addition, medical devices which to date have only had one sensor interface can be equipped according to the invention with a communication interface. Moreover, plugs which have to date been provided for measurement means can be used for connecting to the interface according to the invention. As a result of the fact that according to the invention one and the same interface can be used for two different purposes, the safety of a patient is also automatically ensured, since, in order to communicate, measurement means must be separate from the interface in order to be able to connect a communication line. As mentioned above, this is of particular importance in the case of a software update. Specifically, the medical device inevitably cannot be used for its own purpose, for example measuring, during such an update. In addition, a laptop computer can be connected to the interface in order to be able to carry out a diagnosis of the medical device or a software update.

Specifically, the invention relates to a method of communicating with a medical device, in which an interface is provided to which either measurement means or an external device can be connected and via which measured signals or data are transmitted from the measurement means or the external device to the medical device.

The interface can be operated in two modes: in a measurement mode and in a communication mode. When measurement means are connected it operates in a measurement mode and when an external device is connected it operates in a communication mode. While in the measurement mode signals are received by way of electrodes or sensors from a patient by the medical device via the interface, in the communication mode the interface is used for unidirectional or bidirectional data exchange with an external device.

Preferably, in the communication mode a software update is transferred from a connected external device into the medical device via the interface. However, in the communication mode data can also be transmitted from the medical device to the external device, for example in order to store the transmitted data in the external device on a bulk memory, such as a hard disk, a floppy disk, a writable CD-ROM or a magnetic tape.

In a particularly preferred embodiment, a changeover between measurement mode and communication mode is effected automatically depending on whether measurement means or an external device are or is connected to the interface.

In particular, the automatic changeover can be effected by means of software of the medical device, a switch at the interface or electronically by an operating mode circuit in the medical device.

In a particularly economic embodiment, the interface comprises contacts which can be used both in the measurement mode and in the communication mode. In the extreme case, therefore, no special contacts are provided which are required exclusively for the communication mode. By virtue of the dual function of the contacts, it is therefore possible for contacts to be saved overall, as a result of which the interface and plugs for connecting to the interface can be implemented in a cost-effective manner. In this embodiment, plugs that to date have been provided for the connection of measurement means to the interface can in principle also be used for communication with an external device. Therefore, there is no need to provide any new plugs or lines for a communication connection.

According to a further aspect, the invention relates to an apparatus for communicating with a medical device and a medical device comprising an apparatus for communication, which apparatus comprises an interface that is designed such that either measurement means or an external device can be connected to it and measured signals or data can be transmitted from the measurement means or the external device to the medical device via it. Such an apparatus is suitable in particular for equipping already existing medical devices which have no possibility for communication with external devices. By replacing an existing interface for measurement means with the apparatus according to the invention, a medical device can be expanded to include a communication function in a cost-effective manner. Above all, the measurement means used to date can furthermore be used with the apparatus according to the invention, provided that they are compatible with the interface of the apparatus according to the invention.

In particular, the interface can be designed such that it operates in a measurement mode when measurement means are connected and in a communication mode when an external device is connected.

In a preferred embodiment, the interface can furthermore be designed such that, in the communication mode, a software update can be transmitted from a connected external device into the medical device via the interface. In accordance with FIG. 3, the medical measurement device 54 can implement, for example, a protocol for the software update, as a result of which a host system 52 connected to the interface 56, such as a PC for example, does not need to have the protocol for the software update.

Preferably, the interface is designed such that a changeover between measurement mode and communication mode can be effected automatically.

The automatic changeover can for example be realized in that software of the medical device is designed for the automatic changeover, or a switch at the interface or an operating mode circuit in the medical device is provided for the automatic changeover.

Finally, in a particularly cost-effective embodiment, the interface does not comprise any additional contacts which can be used only in the communication mode.

Further advantages and application possibilities of the present invention emerge from the following description in conjunction with the examples of embodiments shown in the drawings.

In the description, the claims, the abstract and the drawings, the terms and associated reference numerals shown in the attached list of references are used.

The invention will be further described with reference to examples of embodiments shown in the drawings to which, however, the invention is not restricted.

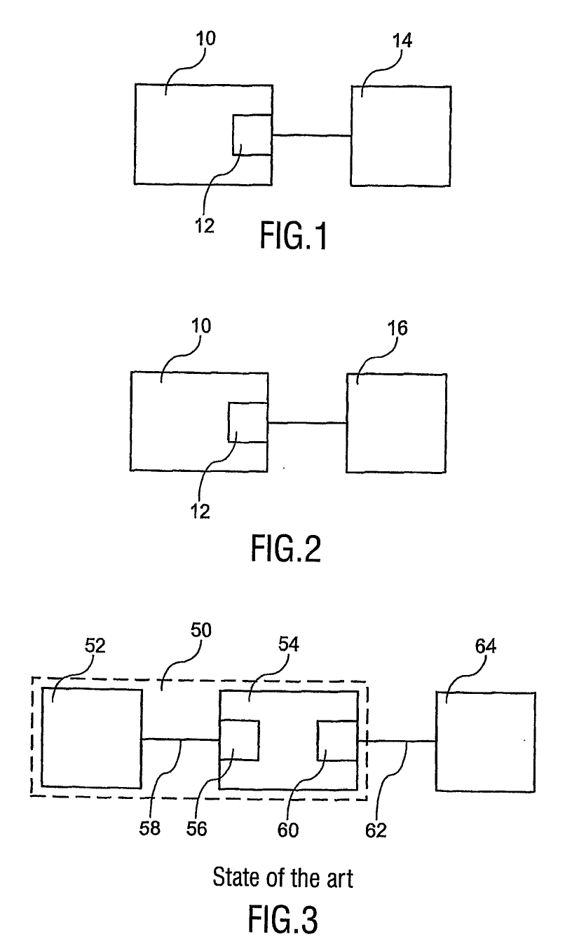

FIG. 1 shows a block diagram of an example of embodiment of the invention, in which a sensor is connected to a medical device.

FIG. 2 shows a block diagram of an example of embodiment of the invention, in which an external device is connected to a medical device.

FIG. 3 shows a block diagram of a conventional medical device, in which a sensor is connected to the medical device via a special interface.

FIG. 4 shows a block diagram of a conventional medical device, in which a sensor is connected to the medical device via a first special interface and a software update device is connected to the medical device via a second special interface.

Hereinbelow, identical, functionally identical and similar elements may be provided with the same reference numerals. Reference is made to the introduction to the description as regards the description of FIGS. 3 and 4.

FIG. 1 shows a medical device 10, for example a portable cardiograph, having an interface 12 to which a sensor 14 is connected as measurement means, said sensor 14 comprising, for example, a number of electrodes for recording an electrocardiogram. The device 10 operates in this configuration in a measurement mode. This means that the device 10, via the interface 12, receives analog electrical signals from the sensor 14, said signals being picked up by the electrodes.

In the measurement mode, the interface 12 is configured such that the received analog signals are forwarded to a processing unit (not shown) that is provided in the device 10, which processing unit in particular converts the analog signals into digital signals and prepares the digital signals for further processing, for example converts them into a predefined format for processing by a PC.

In a communication mode, on the other hand, an external device 16, in particular a PC, is connected to the interface 12. In this operating mode, digital data can be exchanged between the medical device 10 and the external device 16 via the interface 12. Preferably, this operating mode is used to perform a software update in the medical device 10 from the external device 16. This software update comprises the transmission of software from the external device 16 to the medical device 10 via the interface 12, and also the control of the update process.

The same contacts of the interface 12 are used both in the measurement mode and in the communication mode. This means that the interface 12 does not need to have any additional contacts which are provided only for the communication mode. The interface 12 and the associated components, such as the connecting plugs or jacks for example, may therefore be implemented in a cost-effective manner, since only as many contacts need to be provided as are necessary in the measurement mode.

The function of the contacts can be changed over from the measurement mode to the communication mode and vice versa by a mechanical switch. The switch may, for example, be integrated in the interface such that it is not actuated when a line connected to the sensor 14 is connected, but is actuated, on the other hand, when a line to the external device 16 is connected. One possible embodiment of the interface 12 for this purpose could be designed such that the line which is generally connected permanently to the sensor 14, on a plug which can be connected to the interface 12, has a recess in which the switch fitted on the interface is inserted when plug and interface 12 are coupled together, without said switch being actuated. The line for connecting the medical device 10 to an external device 16 may then not have such a recess, in order to reliably close the switch when this line is connected to the interface 12, as a result of which a changeover to the communication mode is effected. The switch may, for example, be a microminiature switch.

A magnetic switch which can be actuated by a magnet incorporated in the plug of the line connected to the sensor 14 is also conceivable. Accordingly, the plug of the line for connecting the external device 16 has no magnets, so that the magnetic switch is not actuated.

The switch can also be implemented in software. By way of example, a software routine may be provided in the medical device 10, which software routine continuously monitors the interface 12. As soon as the routine detects that digital data are being transmitted via the interface 12, it changes over into the communication mode since not digital signals but rather analog signals are transmitted using a sensor. In this case, the external device 16 is connected to the interface 12. However, if analog signals are received via the interface 12, then the routine knows that the sensor 14 is connected to the interface 12 and accordingly activates the measurement mode.

Finally, the changeover between measurement mode and communication mode can also be effected electronically by an operating mode circuit being provided in the medical device, which operating mode circuit measures certain electrical parameters of the signals transmitted via the interface and, in dependence thereon, changes over between the various operating modes. In particular, the operating mode circuit can monitor levels of the transmitted signals and evaluate them. If the levels correspond to those of digital signals, a changeover to the communication mode is effected. Otherwise, the operating mode circuit changes the interface 12 over to the measurement mode.

List of References

10 Medical device
12 Interface
14 Sensor
16 External device
50 Medical device
52 Host system
54 Medical measurement device
56 Communication interface
58 Electrical line
60 Sensor interface
62 Electrical line
64 Sensor
66 PC
68 External interface
70 Electrical line
72 Medical device

1. A method of communicating with a medical device, in which an interface is provided to which either measurement means or an external device can be connected and via which measured signals or data are transmitted from the measurement means or the external device to the medical device.

2. A method as claimed in claim 1, wherein the interface operates in a measurement mode when measurement means are connected and in a communication mode when an external device is connected.

3. A method as claimed in claim 2, wherein in the communication mode a software update is transmitted from a connected external device into the medical device via the interfaces.

4. A method as claimed in claim 2, wherein a changeover between measurement mode and communication mode is effected automatically depending on whether measurement means or an external device are or is connected to the interface.

5. A method as claimed in claim 4, wherein the automatic changeover is effected by means of software of the medical device, a switch at the interface or electronically by an operating mode circuit in the medical device.

6. A method as claimed in claim 1, wherein the interface comprises contacts which can be used both in the measurement mode and in the communication mode.

7. A method as claimed in claim 6, wherein all contacts required for the communication mode can also be used in the measurement mode.

8. An apparatus for communicating with a medical device, which apparatus comprises an interface that is designed such that either measurement means or an external device can be connected to it and measured signals or data can be transmitted from the measurement means or the external device to the medical device via it.

9. A medical device with an apparatus for communication, which apparatus comprises an interface that is designed such that either measurement means or an external device can be connected to it and measured signals or data can be transmitted from the measurement means or the external device to the medical device via it.

10. An apparatus as claimed in claim 8 or a medical device as claimed in claim 9, wherein the interface is designed such that it operates in a measurement mode when measurement means are connected and in a communication mode when an external device is connected.

11. An apparatus or a medical device as claimed in claim 10, wherein the interface is designed such that in the communication mode a software update can be transmitted from a connected external device into the medical device via the interface.

12. An apparatus or a medical device as claimed in claim 10 or 11, wherein the interface is designed such that a changeover between measurement mode and communication mode can be effected automatically.

13. An apparatus or a medical device as claimed in claim 12, wherein software of the medical device is designed for the automatic changeover, or a switch at the interface or an operating mode circuit in the medical device is provided for the automatic changeover.

14. An apparatus or a medical device as claimed in claim 9, wherein the interface comprises contacts which can be used both in the measurement mode and in the communication mode.

15. An apparatus or a medical device as claimed in claim 14, wherein all contacts required for the communication mode can also be used in the measurement mode.
